SEQUENCE LISTING (10 sequences)

SEQ ID NO: 1 - DNA, 57 bp, Artificial Sequence
  Description of Artificial Sequence Synthetic oligonucleotide
tcgattcccagatgcacaggaggagatcgatctcctcctgtgcatctgggaattttt

SEQ ID NO: 2 - DNA, 57 bp, Artificial Sequence
  Description of Artificial Sequence Synthetic oligonucleotide
tcgaatgcacaggaggagaagcaggatcgatcctgcttctcctcctgtgcatttttt

SEQ ID NO: 3 - DNA, 57 bp, Artificial Sequence
  Description of Artificial Sequence Synthetic oligonucleotide
tcgaaagccagtcatggatgaccagatcgatctggtcatccatgactggcttttttt

SEQ ID NO: 4 - DNA, 57 bp, Artificial Sequence
  Description of Artificial Sequence Synthetic oligonucleotide
tcgaatggatgaccagcgcgaccttatcgataaggtcgcgctggtcatccatttttt

SEQ ID NO: 5 - DNA, 57 bp, Artificial Sequence
  Description of Artificial Sequence Synthetic oligonucleotide
tcgacaatgagcaactgcccatgctatcgatagcatgggcagttgctcattgttttt

SEQ ID NO: 6 - DNA, 57 bp, Artificial Sequence
  Description of Artificial Sequence Synthetic oligonucleotide
tcgacctgcagctggagaacctgcgatcgatcgcaggttctccagctgcaggttttt

SEQ ID NO: 7 - DNA, 57 bp, Artificial Sequence
  Description of Artificial Sequence Synthetic oligonucleotide
tcgagcctgtgagcaagatgcgcatatcgatatgcgcatcttgctcacaggcttttt

SEQ ID NO: 8 - DNA, 57 bp, Artificial Sequence
  Description of Artificial Sequence Synthetic oligonucleotide
tcgatgccaccaagtatggcaacatatcgatatgttgccatacttggtggcattttt

SEQ ID NO: 9 - DNA, 57 bp, Artificial Sequence
  Description of Artificial Sequence Synthetic oligonucleotide
tcgacatgacagaggaccatgtgatatcgatatcacatggtcctctgtcatgttttt

SEQ ID NO: 10 - DNA, 57 bp, Artificial Sequence
  Description of Artificial Sequence Synthetic oligonucleotide
tcgacctgagacaccttaagaacacatcgatgtgttcttaaggtgtctcaggttttt

(19) United States
(12) Patent Application Publication
(10) Pub. No.: US 2009/0060889 A1
(43) Pub. Date: Mar. 5, 2009

(54) Ii-RNAi involved Ii suppression in cancer immunotherapy

(76) Inventors: Eric von Hofe, Wellesley, MA (US); Minzhen Xu, Northborough, MA (US)

Correspondence Address: KEVIN FARRELL;PIERCE ATWOOD, ONE NEW HAMPSHIRE AVENUE, PORTSMOUTH, NH, 03801, US

(21) Appl. No.: 12/075,221

(22) Filed: Mar. 10, 2008

Related U.S. Application Data
(60) Provisional application No. 60/906,363, filed on Mar. 12, 2007.

(51) Int. Cl. A61K 48/00; A61K 31/7105; A61K 31/711; A61P 35/00; A61K 35/12; C12N 5/10

(52) U.S. Cl. 424/93.21; 514/44; 435/325; 435/375

(57) ABSTRACT

Provided are compositions and methods involving the inhibition of Ii expression in cells for the purpose of altering antigen presentation pathways. Human Ii-RNAi constructs that effectively inhibit Ii expression in human cancer cells have been generated. The combination of different Ii-RNAi constructs that target different positions of Ii mRNA has a synergistic effect on Ii inhibition. Furthermore, specific promoters for driving Ii-RNAi expression are critical for the activity of Ii-RNAi in different types of cells. Active Ii-RNAi sequences were cloned into plasmids in which Ii-RNAi sequences are driven by either a CMV or an EF-1α promoter. Compositions and methods are disclosed for inhibiting Ii and treating cancer. Provided are siRNAs and expression constructs comprising DNA sequences which encode siRNAs effective to inhibit Ii expression, cells containing such DNA constructs or siRNAs, and methods for use of the same.

### BACKGROUND OF THE INVENTION

The immune response to specific antigens is regulated by the recognition of peptide fragments of those antigens by T lymphocytes. Within an antigen presenting cell, peptide fragments of the processed antigen become bound into the antigenic peptide binding site of major histocompatibility complex (MHC) molecules. These peptide-MHC complexes are then transported to the cell surface for recognition (of both the foreign peptide and the adjacent surface of the presenting MHC molecule) by T cell receptors on helper or cytotoxic T lymphocytes. There are two classes of MHC molecules that deliver peptides, MHC class I and MHC class II.

MHC class I molecules present antigen to CD8-positive cytotoxic T-lymphocytes (T killer cells), which then become activated and can kill the antigen presenting cell directly. Class I MHC molecules exclusively receive peptides from endogenously synthesized proteins, such as an infectious virus, in the endoplasmic reticulum at around the time of their synthesis.

MHC class II molecules present antigen to CD4-positive helper T-lymphocytes (T helper cells). Once activated, T helper cells contribute to the activation of cytotoxic T lymphocytes and B lymphocytes via physical contact and cytokine release. Unlike MHC class I molecules, MHC class II molecules bind exogenous antigens which have been internalized via non-specific or specific endocytosis. The MHC Class II associated invariant chain protein (Ii) normally blocks the processing of endogenous peptides, preventing attachment to MHC and antigen presentation.

Another major function of the Ii protein is to enhance exogenous peptide charging to MHC class II molecules (Xu, M., et al., Mol Immunol 31:723-731 (1994); Daibata, M., et al., Mol Immunol 31:255-260 (1994); Reyes, V. E., et al., Ann N Y Acad Sci 730:338-341 (1994)). The Ii protein normally binds to MHC class II molecules in the endoplasmic reticulum at synthesis and protects the epitope-binding site on MHC class II molecules from binding to endogenously-derived epitopes in the endoplasmic reticulum, as normally occurs with MHC class I molecules (Bertolino, P. and Rabourdin-Combe, C., Crit Rev Immunol 16:359-379 (1996); Bodmer, H., et al., Science 263:1284-1286 (1994)). These MHC class II-Ii protein complexes are transported from the endoplasmic reticulum to a post-Golgi, antigenic peptide-binding compartment where Ii is released by proteolysis and exogenous antigenic peptides are bound (Daibata et al., Molecular Immunology 31: 255-260 (1994); Xu et al., Molecular Immunology 31: 723-731 (1994); Bakke, O. and Dobberstein B., Cell 63:707-716 (1990); Lamb, C. A. and Cresswell, P., J Immunol 148:3478-3482 (1992); Blum, J. S. and Cresswell, P., Proc Natl Acad Sci USA 85:3975-3979 (1988)). In such compartments, the Ii is cleaved by proteases to allow charging by exogenously derived epitopes. After being charged with epitopes, the MHC class II/epitope complex travels to the cell surface for presentation to CD4+ Th cells (Nguyen, Q. V et al., Hum Immunol 24:153-163 (1989); Shi, G. P., et al., J Exp Med 191:1177-1186 (2000); Riese, R. J., et al., Immunity 4:357-366 (1996); Hiltbold, E. M. and Roche, P. A., Curr Opin Immunol 14:30-35 (2002)).

Under normal conditions, endogenous peptides (with self determinants potentially leading to autoimmune disease) are not bound to MHC class II molecules since the Ii protein is always cosynthesized with nascent MHC class II molecules. Because complexes containing autodeterminant peptides and MHC class II molecules are never seen by the body's immune surveillance system, tolerance is not developed to these determinants. If MHC class II molecules are not inhibited by Ii in a developed individual, endogenous autodeterminants then become presented by MHC class II molecules, initiating an autoimmune response to those endogenous antigens. Such is the case in certain autoimmune diseases. By engineering such an effect in malignant cells, an “autoimmune response” to the endogenous antigens of a tumor can be used therapeutically to either restrict growth or eliminate tumor cells.

Suppression of the Ii protein in tumor cell lines and rat tumor models has been shown to induce tumor antigen presentation and enhance antigen specific tumor cell killing. The therapeutic effects of increased MHC class II molecule expression without concomitant increase in Ii protein has been demonstrated in MHC class II-negative, Ii-negative tumors (Ostrand-Rosenberg et al., Journal of Immunol. 144: 4068-4071 (1990); Clements et al., Journal of Immunol. 149: 2391-2396 (1992); Baskar et al., Cell. Immunol. 155: 123-133 (1994); Baskar et al., J. Exp. Med. 181: 619-629 (1995); and Armstrong et al., Proc. Natl. Acad. Sci. USA 94: 6886-6891 (1997)). In these studies, transfection of genes for MHC class II molecules into a MHC class II-negative murine sarcoma generated MHC class II-positive, but Ii-negative, tumor cell lines. Injection of these cells into a MHC compatible host led to the delayed growth of the parental tumors. Co-transfection of the gene for the Ii protein into a sarcoma cell line, along with the MHC class II genes, inhibited the tumor-therapeutic effect of the MHC class II genes since the Ii chain blocked the presentation of endogenous tumor antigens. Comparable results have been produced with a murine melanoma (Chen and Ananthaswamy, Journal of Immunology 151: 244-255 (1993)).

The success of this therapeutic approach is thought to involve the natural activities of dendritic cells. Dendritic cells are professional scavengers, which process foreign antigens into peptides and present them to T lymphocytes from MHC antigens on their cell surfaces. Dendritic cells have the capacity to present antigen through both MHC class I and class II molecules, enabling them to activate both T helper and T killer cells. It is thought that an effective T helper cell response is required to elicit a powerful T killer cell response and that the combined activation produced by dendritic cells leads to a heightened anti-tumor response (Ridge et al., Nature 193: 474-477 (1998); Schoenberger et al., Nature 193: 480-483 (1998)). The dendritic cells of macrophage lineage, upon finding tumor cells, ingest and process both tumor-specific and tumor-related antigens. The dendritic cells then migrate to the lymph nodes which drain the tumor site and reside in those nodes near the node cortex where new T cells germinate. In the node cortex, resting T killer cells which recognize tumor determinants on the dendritic cells, become activated and proliferate, and are subsequently released into the circulation as competent, anti-tumor, killer T cells.

Although interaction with T-helper cells activates or “licenses” dendritic cells to present antigen through MHC class I molecules, and hence to activate T killer cells, simultaneous interaction with T helper cells and T killer cells is not necessary; activated dendritic cells maintain their capacity to stimulate T killer cells for some time after T helper cell mediated activation. The respective antigenic peptides which become presented by either MHC class II or MHC class I determinants do not need to come from one antigenic protein, two or more antigens from a malignant cell can be processed and presented by a dendritic cell. Therefore, licensing to one determinant, perhaps not tumor specific, carries with it the power to license activation of T killer cells to other, perhaps tumor-specific, determinants. Such ‘minor’ or ‘cryptic’ determinants have been used for various therapeutic purposes (Mougdil et al., J. Immunol. 159: 2574-2579 (1997)).

Experimental alteration of MHC class II antigen presentation is thought to expand immune responses to these minor determinants. This series of peptides usually unavailable for charging to MHC class II molecules provides a rich source of varied peptides for MHC class II presentation. Exploitation of this series of determinants leads to the expansion of populations of responsive T helper cells. Such expanded populations can elicit dendritic cell licensing, some of which are directed toward tumor specific and tumor related determinants. Although normal cells potentially share tumor cell determinants, only minor cellular damage occurs to normal cells. This is because the multiple effector responses (mass of killer T cells, ambient activating cytokines, phagocytosing macrophages and their products, etc.) of the anti-tumor response are not directed toward normal cells.

Normal MHC class II antigen presentation can be altered by inhibiting the interactions of MHC class II molecules with the Ii protein. This is accomplished by decreasing total Ii protein, (e.g. by decreasing expression) or by otherwise interfering with the Ii immunoregulatory function. Inhibition of Ii expression has been accomplished using various antisense technologies. An antisense oligonucleotide interacting with the AUG site of the mRNA for Ii protein has been described to decrease MHC class II presentation of exogenous antigen (Bertolino et al., Internat. Immunology 3: 435-443 (1991)). However, the effect on the expression of Ii protein and on the presentation of endogenous antigen by MHC class II molecules was not examined. More recently Humphreys et al., in U.S. Pat. No. 5,726,020 (1998), identified three antisense oligonucleotides and a reverse gene construct which upon introduction into an antigen presenting cell expressing MHC class II molecules effectively suppresses Ii protein expression. Mice inoculated with tumor cells which are Ii suppressed by this mechanism were shown to survive significantly longer than mice inoculated with the untreated parent tumor cells. This observation indicates that the suppression of Ii protein generated an increase in range of antigenic determinant presentation, and thus triggered a more effective immune response to the tumor cells.

In the sarcoma cell (Sal1) tumor model, tumor cells treated with this Ii antisense oligonucleotide are potent vaccine against challenge by parental tumor. As clinically useful in vivo therapeutic antisense reagents, expressible Ii antisense reverse gene constructs (Ii-RGC) were created (U.S. patent application Ser. No. 10/127,347). These were constructed by cloning different Ii gene fragments in reverse orientation into expressible plasmids or adenoviruses, to evaluate multiple methods of tumor cell administration (Hillman et al., Gene Ther. 10, 1512-8 (2003); Hillman et al., Human Gene Therapy 14, 763-775 (2003)). The Ii-RGC genes were evaluated by DNA transfections in several murine tumor cell lines, including A20 lymphoma cells, MC-38 colon adenocarcinoma cells, Renca renal adenocarcinoma cells, B16 melanoma cells, and RM-9 prostate cancer cells. The most active one, Ii-RGC (−92, 97) (A in the AUG start codon is position 1), was used for in vivo studies.

Among the cell lines tested, A20 is already MHC class II+/Ii+. Ii-RGC (−92, 97) significantly inhibited Ii expression when this construct was delivered by lipid or gene gun transfection methods. The other tumor lines tested are MHC class II−/Ii−. These cell lines were co-transfected in vitro with Ii-RGC (−92, 97) and either CIITA or IFN-γ, or both, creating the MHC class II-positive/Ii-suppressed phenotype (Lu et al., Cancer Immunol Immunother 48, 492-8 (2003); Hillman et al., Gene Ther. 10, 1512-8 (2003); Hillman et al. Human Gene Therapy 14, 763-775 (2003)). In vivo induction of the MHC class II-positive/Ii-suppressed phenotype was also generated by intratumoral injection of Ii-RGC and CIITA plasmids with lipid (Lu et al., Cancer Immunol Immunother 48, 492-8 (2003); Hillman et al., Human Gene Therapy 14, 763-775 (2003)) or recombinant adenoviral vectors containing Ii-RGC(−92, 97), CIITA and IFN-γ (Hillman et al., Gene Ther. 10, 1512-8 (2003)).

The in vivo activities of these therapeutic constructs were tested by intratumoral injection in established subcutaneous tumors using two tumor models: the Renca renal carcinoma and the RM-9 prostate carcinoma. In both tumor models, complete regression of established tumors was achieved. In the Renca model, tumor regression was observed in about 50% of mice following four intratumoral injections of CIITA and Ii-RGC plasmid constructs over 4 days given together with a suboptimal dose of IL-2 plasmid (Lu et al., Cancer Immunol Immunother 48, 492-8 (2003)). Intratumoral injections of recombinant adenovirus, containing CIITA, IFN-γ, Ii-RGC constructs and IL-2 gene, in established Renca tumors induced complete tumor regression in about 60-70% of mice and protection against Renca tumor rechallenge (Hillman et al., Gene Ther. 10, 1512-8 (2003)). In an aggressive, poorly immunogenic RM-9 prostate tumor model, radiation augmented the effect of the suboptimal dose of IL-2 and MHC class II-positive/Ii-suppressed phenotype causing complete tumor regression in 50% of the mice (Hillman et al., Human Gene Therapy 14, 763-775, 2003). Established RM-9 subcutaneous tumors were selectively irradiated and treated a day later with intratumoral plasmid gene therapy using the plasmids pCIITA, pIFN-γ, pIL-2 and pIi-RGC for four consecutive days. Intratumoral treatment with all the four plasmids induced complete tumor regression in more than 50% of the mice only when tumor irradiation was administered one day prior to gene therapy. Mice rendered tumor-free by radiation and intratumoral gene therapy and re-challenged on day 64 were protected against RM-9 challenge but not against syngeneic EL4 challenge. The findings demonstrated that in the RM-9 model, radiation enhanced the therapeutic efficacy of intratumoral gene therapy for in situ induction of tumor-specific immune response.

In order to obtain optimal therapeutic effect, MHC class II and Ii must be induced with CIITA and Ii needs to be inhibited by Ii-RGC in both the Renca and RM-9 tumor models (Lu et al., Cancer Immunol Immunother 48, 492-8 (2003); Hillman et al., Human Gene Therapy 14, 763-775, 2003). The results are consistent with those of Martin et al. (J Immunol 162, 6663-70 (1999)) who showed, in a murine lung carcinoma model, that induction of MHC class II by CIITA did not create an efficient tumor cell vaccine. This study confirms the finding that induction of MHC class II by transfecting CIITA, which also induces Ii, is insufficient for a therapeutic effect. One must obtain the therapeutic phenotype of MHC class II+/Ii− by also suppressing Ii protein. In order to test for optimal suppression of Ii protein, the therapeutic constructs CIITA and Ii-RGC were used at different ratios. At least a 1:4 ratio (CIITA:Ii-RGC) was required to ensure good inhibition of Ii. IFN-γ is used in the RM-9 prostate tumor to induce MHC class 1 molecules which are not expressed in the parental cells. Renca cells are MHC class I-positive cells and IFN-γ is not needed to induce MHC class 1 molecules but does upregulate further their expression. In both tumor models, a subtherapeutic dose of IL-2 plasmid is needed to promote the immune response.

Given this clear demonstration of efficacy in curing established tumors in mice, and steady progression in preclinical studies to determine optimal treatment protocols, reagents for treating human cancers were created. The CIITA gene used in the mice studies is human and its product functions well on the murine promoters for MHC class II and Ii genes (Ting et al., Cell 109, 521-33 (1999)). Several human Ii-RGCs, which inhibited Ii expression in a human B lymphoblastoid and the HeLa cell lines were created. Transduction of cells with CIITA construct induced upregulation of cell surface MHC class II molecules and intracellular Ii, while transduction of cells with both CIITA and hIi-RGC caused suppression of Ii without affecting enhanced expression of MHC class II. These data were reproduced in additional human tumor cell lines including the human B lymphoma cell line Raji, and human melanoma cell line.

In U.S. application Ser. No. 10/999,208, filed Nov. 29, 2004, incorporated herein by reference, these methods were applied with newly designed RNA interference (RNAi) genetic constructs and synthetic oligonucleotides. Double stranded RNA (dsRNA) can be used for selective inhibition of target gene expression by RNAi in mammalian cells. Unlike antisense, RNAi is mediated by the incorporation of double stranded RNA into a nuclease complex, termed the RNA-induced silencing complex (RISC) that subsequently cleaves the target RNA. It has been shown that double stranded RNAs less than 25 nucleotides in length do not activate an RNA response characteristic of viral infections. The RNA sequences can be based on any region of the target gene RNA, generally in the coding region. When using synthetic RNAi, cells are treated in culture using cationic lipids for delivery of nanomolar concentrations of RNAi. Active RNAi may also be engineered into expression constructs. RNAi not complimentary to the target sequence is used as a control. Inhibition of gene expression is measured 12 to 72 hours after RNAi treatment using Western, FACS and/or phenotypic assays.

RNAi is a process by which dsRNA specifically suppresses the expression of a gene bearing its complementary sequence (Moss, Curr. Biol. 11: R772-5 (2001); Elbashir, Genes Dev. 15: 188-200 (2001)). Several gene products have been implicated in this process, including DICER, which is an Rnase that processively cleaves long dsRNA into double-stranded fragments between 21 and 25 nucleotides long. These fragments are known in the art as short interfering or small interfering RNAs (siRNA) (Elbashir et al., 2001).

Studies in Drosophila have shown that DICER processes long dsRNA into siRNAs comprised of two 21 nt strands which includes a 19 nt region on each precisely complementary with the other, yielding a 19 nt duplex region flanked by 2 nt-3′ overhangs (WO 01/75164; Bernstein et al., Nature 409:363, 2001). SiRNAs then induce formation of a protein complex that recognizes and cleaves target mRNAs. Homologs of the DICER enzyme have been identified in species ranging from E. coli to humans (Sharp, 2001; Zamore, Nat. Struct. Biol. 8:746, 2001), suggesting that siRNAs have the ability to silence gene expression in many different cell types including mammalian and human cells.

Subsequently it was discovered that RNAi can be triggered in mammalian cells by introducing synthetic 21-nucleotide siRNA duplexes (Elbashir et al., 2001). In mammalian cell culture, RNAi has been successfully recreated in a wide variety of different cell types with synthetic siRNAs introduced into cells by techniques such as transfection (Elbashir et al., 2001). Because 21 nucleotide siRNAs are too short to induce an interferon response in mammalian cells (Kumar and Carmichael, 1998), but yet long enough to provide sequence specific inhibition of a targeted gene, they possess tremendous potential as research tools and therapeutics.

The robust nature of RNAi inhibition of Ii is ideally suited for immune stimulation resulting from the presentation of endogenously synthesized antigens. Ii only needs to be suppressed in a fraction of the cells for a short period of time to obtain immune stimulation. This is in stark contrast to other specific targets related to the growth of cancer cells requiring continuous inhibition in virtually all cells.

Applicants have previously filed and prosecuted patent applications disclosing Ii inhibition for the purpose of modulating the immune response. These applications specifically disclose inhibitory copolymers which are introduced into a cell and which directly inhibits Ii synthesis by binding to the Ii mRNA, as well as reverse gene constructs which are introduced into a cell as a nucleic acid construct which is subsequently transcribed into an RNA molecule which inhibits Ii expression after specific hybridization. These earlier filed patent applications include U.S. application Ser. Nos. 08/661,627, 09/205,995, 10/054,387 and 10/127,347, the disclosures of which are incorporated herein by reference. U.S. application Ser. Nos. 08/661,627 and 09/205,995 have issued as U.S. Pat. Nos. 5,726,020 and 6,368,855, respectively. U.S. application Ser. No. 10/054,387 is abandoned.

As mentioned briefly above, U.S. application Ser. No. 09/205,995 contains extensive disclosure relating to chemically synthesized copolymers containing from about 10 to about 50 nucleotide bases. These copolymers contain nucleotide base sequences which are complementary to a targeted portion of the RNA molecule, otherwise known as antisense sequences. Examples of such copolymers include antisense oligonucleotides and siRNAs. Antisense copolymers inhibit protein translation from RNA by two mechanisms. One method is to block access to portions of the RNA which must interact with ribosomes, spliceosomes or other factors essential for RNA maturation or translation. A second method, involves potentiation of an enzyme, ribonuclease H, which cleaves sequences of RNA hybridized to DNA. Thus, the binding of a DNA or DNA like copolymer to a corresponding segment in the RNA leads to cleavage of the RNA at the copolymer binding site.

Such oligonucleotide modifications and the characteristics which are produced are readily available to one of skill in the art. Exemplary modifications are presented in U.S. Pat. No. 4,469,863 (1984); U.S. Pat. No. 5,216,141 (1993); U.S. Pat. No. 5,264,564 (1993); U.S. Pat. No. 5,514,786 (1996); U.S. Pat. No. 5,587,300 (1996); U.S. Pat. No. 5,587,469 (1996); U.S. Pat. No. 5,602,240 (1997); U.S. Pat. No. 5,610,289 (1997); U.S. Pat. No. 5,614,617 (1997); U.S. Pat. No. 5,623,065 (1997); U.S. Pat. No. 5,623,070 (1997); U.S. Pat. No. 5,700,922 (1997); and U.S. Pat. No. 5,726,297 (1998), the disclosures of which are incorporated herein by reference.

HLA-DR-associated invariant chain (Ii) is involved in antigen presentation and is expressed on antigen presenting cells. Ii is also expressed by cancer cells, yet its expression is restricted in normal tissue. This observation makes Ii a worthwhile therapeutic target for cancer treatment.

To enhance the effectiveness of immunoatherapeutic tumor cell vaccines, Applicants have developed methods to suppress expression of the MHC class II-associated invariant chain (Ii protein) in tumor cells. This protein normally blocks the antigenic peptide binding site of MHC class II molecules immediately after synthesis. Inhibition of the Ii protein leads to the simultaneous presentation of tumor antigens by both MHC class I and II molecules leading to the activation of both CD4+ and CD8+ T cells, and thus generating a robust and long-lasting anti-tumor immune response.

### SUMMARY

The present invention is directed toward compositions and methods involving the inhibition of Ii expression in cells for the purpose of altering antigen presentation pathways. The present invention relates in one aspect to siRNAs effective to inhibit Ii expression. The siRNAs used in the methods and compositions of this invention each comprise in a single molecule a sense sequence of Ii, a reverse complement of said sense sequence, and an intervening sequence enabling duplex formation between the sense and reverse complement sequences. In another aspect, the present invention provides DNA sequences which encode siRNAs effective to inhibit Ii expression, cells containing such DNAs or siRNAs, and methods for use of the same.

In one aspect, the invention relates to a method for inhibiting expression of Ii in a cell. This method comprises introducing two different siRNAs into a cell expressing Ii, wherein the siRNAs are introduced either directly or indirectly into the cell. The siRNAs thereafter form RNA-induced silencing complexes, thereby inhibiting expression of Ii in the cell.

Human Ii-RNAi constructs that effectively inhibit Ii expression in human Raji (B cell lymphoma), AML, prostate cancer, and Human Embryonic Kidney (HEK)-293 kidney cells have been generated. The instant application describes, for the first time, the suppression of Ii using certain single siRNAs in various human cancer cells. Also disclosed is the significantly greater effect in suppressing Ii when these siRNAs are used in combination. The combination of different Ii-RNAi constructs that target different positions of Ii mRNA has a synergistic effect on Ii inhibition. The percentage of cells in which Ii was inhibited was 65% when using a combination of Ii-RNAi constructs versus 35% when using an equivalent amount of a single Ii-RNAi construct.

In another aspect, the present invention relates to a method for targeting a type of cell of an animal for an immunological response, the type of cell being characterized by the expression of one or more known or unknown antigen(s). In this method a culture of peripheral blood mononuclear cells from an individual is provided, the culture including antigen presenting cells. One or preferably two different siRNA inhibitors of Ii expression are introduced either directly or indirectly into the antigen presenting cells of the culture, such as by one or more expressible nucleic acid sequence(s) encoding the siRNAs into the cells in the culture under conditions appropriate for expression.

Further, specific promoters for driving Ii-RNAi expression are critical for the activity of Ii-RNAi in different types of cells. Active Ii-RNAi sequences were cloned into pBudCE4.1 plasmids in which Ii-RNAi sequences are driven by an EF-1α promoter. Transfection of a human progenitor myeloid leukemia cell line, KG-1, with pBudCE4.1/Ii-RNAi constructs indicates that the EF-1α promoter is more active than a CMV promoter in KG-1 cells, as indicated by greater Ii inhibition.

A practical advantage of this strategy for clinical use is the monomorphic nature of the Ii gene. One-Ii-RNAi construct will be sufficient for all patients with different HLA-DR alleles. The successful generation of the MHC class II+/Ii− phenotype in cancer cells has paved the way for clinical trials using Ii-suppressed cancer cells in patients with AML, prostate cancer, and B cell lymphoma. A number of related aspects are described in detail in the following sections.

### DETAILED DESCRIPTION OF THE INVENTION

The suppression of Ii expression is intended to alter antigen presentation pathways. More specifically, the inhibition of Ii expression is intended to promote the charging of MHC Class II molecules with antigenic epitopes which normally would not be presented in this context. The subject invention relates to compositions and methods involving the inhibition of Ii expression in cells for the purpose of altering antigen presentation pathways.

A required element relating to all aspects of the present disclosure is the inhibition of Ii synthesis in a cell. The term “inhibition” or “suppression” is intended to mean down regulation, or the act of reducing the activity of Ii or the level of Ii RNAs below that observed in the absence of an inhibitor or suppressor of the present invention. As discussed in the Background of the Invention section, Ii is a protein, which is co-regulated with the MHC Class II molecules. Ii binds MHC Class II molecules thereby blocking access to MHC Class II molecules of endogenously synthesized antigens (i.e., antigen synthesized within the MHC Class II molecule-expressing cells). The MHC Class II molecule/Ii complexes are transported from the endoplasmic reticulum to a post-Golgi compartment where Ii is released by a staged cleavage process which enables charging by exogenous antigen (i.e., antigen which is not synthesized within the antigen presenting cell and has been selected for uptake into the antigen presenting cell by mechanisms such as phagocytosis, opsonization, cell surface antibody recognition, complement receptor recognition, and Fc receptor recognition).

Antigens excluded from binding to MHC Class II molecules in the endoplasmic reticulum by virtue of the presence of complexed Ii protein can be referred to as endogenously synthesized antigens. Such antigens comprise a survey of cytoplasmic proteins, which have been digested by proteosomes and transported as peptides into the endoplasmic reticulum by the transporter of antigenic peptides (TAP). Such endogenously synthesized antigens are normally bound to MHC Class I molecules in the endoplasmic reticulum. Such antigenic fragments are not normally bound in the endoplasmic reticulum to MHC Class II molecules because Ii protein blocks the antigenic peptide-binding site.

By suppressing the expression of the Ii protein, this vast repertoire of peptides, which have been transported into the endoplasmic reticulum for binding to MHC Class I molecules and subsequent presentation to CD8+ T lymphocytes, can bind to MHC Class II molecules for subsequent presentation to, and activation of, CD4+ T immunoregulatory cells. CD4+ T immunoregulatory cells can have either helper or suppressor functions in orchestrating various pathways of the immune response. They contribute to the activation of other cells, such as cytotoxic T lymphocytes (T killer cells), B lymphocytes, and dendritic cells, via physical contact and cytokine release.

The term “antigenic epitope of interest,” as used herein, refers to an antigenic epitope present in a peptide derived from a protein produced within the cell on which antigen presentation is to take place. The term, as used herein, is intended to encompass antigenic epitopes which are known or unknown. Thus the modifier “of interest” does not imply that the epitope is predetermined. An antigenic epitope is “of interest” merely because it is contained in a protein which is synthesized in the cytoplasm of the cell on which presentation is to take place.

A significant biological consequence, offering an opportunity for therapeutic intervention, follows from the binding by MHC Class II molecules of peptides, from the repertoire of peptides transported into the endoplasmic reticulum for binding there by MHC Class I molecules. Often the epitopes bound to the MHC Class II molecules in the presence of Ii suppression are “cryptic” epitopes in that such epitopes are not otherwise presented to the immune system in association with MHC Class II molecules by classical pathways of antigen presentation. Cryptic epitopes can be revealed experimentally by analyzing a library of overlapping synthetic peptides of the amino acid sequence of a test antigen. Animals of one strain of mice immunized with the test antigen can be found to respond to a set of peptides from the library (the “dominant epitopes”). However, when otherwise identical mice are immunized with single peptides of the library, a previously unidentified subset (in addition to any dominant epitopes in the immunizing peptide) is found to contain immunological epitopes. These previously unidentified epitopes comprise a set of cryptic epitopes.

Although the methods of this invention promote immunity against both dominant and cryptic epitopes, in some clinical situations the enhancement of the immune response to cryptic epitopes plays a special role in the therapeutic effect. For example, in the case of boosting a therapeutic response to cancer-related antigenic epitopes, a T helper cell response to cryptic epitopes to which a suppressor T cell response has never occurred is more likely to provide for effective dendritic cell licensing which, in turn, creates a robust cytotoxic T lymphocyte anti-tumor response. The development of suppressing T cell responses to dominant epitopes of cancer-related antigens has been shown to play a role in the growth of tumor micrometastases. A significant utility of this invention is therefore promotion of T helper cell responses to putatively cryptic cancer-related determinants.

A variety of antigens which fall within the “endogenously synthesized” class (which are normally excluded from MHC Class II molecule presentation) are specifically associated with certain pathological conditions. Consider, for example, tumor cells or other malignant cells. Such cells synthesize cancer-specific and cancer-related proteins, which contain therapeutically useful MHC Class II epitopes. However, because these proteins are synthesized within the antigen presenting cell, antigenic epitopes of such proteins are excluded from presentation in association with MHC Class II molecules of the same cell. The ability to alter the normal course of events, thereby presenting pathology-specific antigen in association with MHC Class II molecules, results in enhancement of responses initiated by novel MHC Class II antigenic epitopes.

An array of therapeutic modalities fall within the scope of the present invention. Patentable compositions are associated with many of these therapeutic modalities. Therapeutic approaches include in vivo and ex vivo embodiments. It is an object of the present invention to provide compositions comprising one or more siRNAs effective to inhibit Ii expression, and also to provide vectors and cells containing such compositions, and methods of use for the same.

Double-stranded siRNAs, and genes encoding these molecules, may be used to inhibit Ii by RNA interference. The term “RNA interference (RNAi)” as used herein refers to the process by which dsRNA specifically suppresses the expression of a gene bearing its complementary sequence (Moss, Curr. Biol. 11(19): R772-5 (2001); Elbashir, Genes Dev. 15(2): 188-200 (2001)). While not wishing to be bound by theory, RNAi is understood to occur by a mechanism involving multiple RNA-protein interactions, characterized by four major steps: assembly of siRNA with the RNA-induced silencing complex (RISC), activation of the RISC, target recognition, and target cleavage. The term “short interfering RNA (siRNA)” as used herein is intended to refer to any nucleic acid molecule capable of mediating RNAi or gene silencing. The term siRNA is intended to encompass various naturally generated or synthetic compounds, with RNAi function. Such compounds include, without limitation, duplex synthetic oligonucleotides, of about 21 to 23 base pairs with terminal overlaps of 2 or 3 base pairs; hairpin structures of one oligonucleotide chain with sense and complementary, hybridizing, segments of about 21-23 base pairs joined by a loop of about 3-5 base pairs; and various genetic constructs leading to the expression of the preceding structures or functional equivalents. Such genetic constructs are usually prepared in vitro and introduced in the test system, but can also include siRNA from naturally occurring siRNA precursors coded by the genome of the host cell or animal.

It is not a requirement that an siRNA of the present invention be comprised solely of RNA. An siRNA of the present invention may comprise one or more chemical modifications and/or nucleotide analogues. The modification and/or analogue may be any modification and/or analogue, respectively, that does not negatively affect the ability of the siRNA to inhibit Ii expression. The inclusion of one or more chemical modifications and/or nucleotide analogues in an siRNA may be preferred to prevent or slow nuclease digestion and, in turn, create a more stable siRNA for practical use. Chemical modifications and/or nucleotide analogues which stabilize RNA are known in the art. Phosphorothioate derivatives, which include the replacement of non-bridging phosphoroyl oxygen atoms with sulfur atoms, are one example of analogues showing increased resistance to nuclease digestion. Sites of the siRNA which may be targeted for chemical modification include the loop region of a hairpin structure, the 5′ and 3′ ends of a hairpin structure (e.g. cap structures), the 3′ overhang regions of a double-stranded linear siRNA, the 5′ or 3′ ends of the sense strand and/or antisense strand of a linear siRNA, and one or more nucleotides of the sense and/or antisense strand. As used herein, the term siRNA is intended to be equivalent to any term in the art defined as a molecule capable of mediating sequence-specific RNAi. Such equivalents include, for example, double-stranded RNA (dsRNA), micro-RNA (mRNA), short hairpin RNA (shRNA), short interfering oligonucleotide, and post-transcriptional gene silencing RNA (ptgsRNA).

While not wishing to be bound by theory, it is generally understood that in RNAi dsRNA is processed into 21 to 23 base-pair fragments that bind to and lead to the degradation of the complementary mRNA (Bernstein, Nature 409(6818): 363-6 (2001), and International Publication Number WO 0175164). These siRNAs induce sequence-specific posttranslational gene silencing. Such molecules may be introduced into cells to suppress gene expression for therapeutic or prophylactic purposes as described in International Publication Number WO 0175164 and in various patents, patent applications and papers. Publications herein incorporated by reference, describing RNAi technology include but are not limited to the following: U.S. Pat. No. 6,686,463, U.S. Pat. No. 6,673,611, U.S. Pat. No. 6,623,962, U.S. Pat. No. 6,506,559, U.S. Pat. No. 6,573,099, and U.S. Pat. No. 6,531,644; International Publication Numbers WO04061081; WO04052093; WO04048596; WO04048594; WO04048581; WO04048566; WO04046320; WO04044537; WO04043406; WO04033620; WO04030660; WO04028471; WO 0175164. Papers which describe the methods and concepts for the optimal use of these compounds include but are not limited to the following: Brummelkamp Science 296: 550-553 (2002); Caplen Expert Opin. Biol. Ther. 3:575-86 (2003); Brummelkamp, Sciencexpress 21 Mar. 3 1-6 (2003); Yu Proc Natl Acad Sci USA 99:6047-52 (2002); Paul Nature Biotechnology 29:505-8 (2002); Paddison Proc Natl Acad Sci USA 99:1443-8 (2002); Brummelkamp Nature 424: 797-801 (2003); Brummelkamp, Science 296:-550-3 (2003); Sui Proc Natl Acad Sci USA 99: 5515-20 (2002); Paddison, Genes and Development 16:948-58 (2002).

In the context of the present invention, a composition comprising an siRNA effective to inhibit Ii expression may include an RNA duplex comprising a sense sequence of Ii. In this embodiment, the RNA duplex comprises a first strand comprising a sense sequence of Ii and a second strand comprising a reverse complement of the sense sequence of Ii.

In another embodiment, a composition comprising an siRNA effective to inhibit Ii expression may comprise in a single molecule a sense sequence of Ii, the reverse complement of the sense sequence of Ii, and an intervening sequence enabling duplex formation between the sense and reverse complement sequences. An siRNA of the present invention may comprise the RNA of a sequence selected from the group consisting of SEQ ID NO: 14, SEQ ID NO: 16, and SEQ ID NO: 17.

It will be readily apparent to one of skill in the art that an siRNA of the present invention may comprise a sense sequence of Ii or the reverse complement of the sense sequence of Ii which is less than perfectly complementary to each other or to the targeted region of Ii. In other words, the siRNA may comprise mismatches or bulges within the sense or reverse complement sequence. In one aspect, the sense sequence or its reverse complement may not be entirely contiguous. The sequence or sequences may comprise one or more substitutions, deletions, and/or insertions. The only requirement of the present invention is that the siRNA sense sequence possesses enough complementarity to its reverse complement and to the targeted region of Ii to allow for RNAi activity. It is an object of the present invention, therefore, to provide for sequence modifications of an siRNA of the present invention that retain sufficient complementarity to allow for RNAi activity. One of skill in the art may predict that a modified siRNA composition of the present invention will work based on the calculated binding free energy of the modified sequence for the complement sequence and targeted region of Ii. Calculation of binding free energies for nucleic acids and the effect of such values on strand hybridization are known in the art.

U.S. application Ser. No. 10/999,208 describes the alteration of antigen presentation pathways through the use of single Ii siRNAs effective to inhibit Ii expression and through the use of DNA sequences which encode single siRNAs effective to inhibit Ii expression. The inhibition was demonstrated in HEK-293, non-cancerous cells. Cells containing such single siRNAs or the DNA which encodes them and methods for their use were also described.

The present invention is based, in part, on the discovery that three of these siRNAs inhibit Ii in human cancer cells and furthermore that when any of these siRNAs are used in combination, the inhibition of Ii is greatly increased compared to the effect produced by single siRNAs. As shown in the Example 1 of the Exemplification section, single siRNA SEQ ID NOS: 14, 16, and 17 inhibited Ii 27-32.5% compared to the control. This is the first time inhibition of Ii was demonstrated in cancer (Raji) cells with single or a combination of siRNAs.

In one embodiment, the present invention includes a method of suppressing Ii in a cell comprising administering any two different siRNAs of the group consisting of SEQ ID NOS: 14, 16, and 17. The methods of the invention include administering any two of these three siRNA sequences, especially the combination of SEQ ID NOS: 14 and 17, as this combination produced the greatest effect in inhibiting Ii in Raji cells (Table 3).

As is detailed in the Exemplification section, Ii was inhibited by the methods and compositions of the present invention in Raji (B cell lymphoma), AML, and prostate cancer cells. It is discussed in the Background section that Ii suppression is a target of cancer immunotherapy and that suppressing Ii can trigger a more effective immune response to tumor cells. Thus, in one embodiment, the present invention includes methods of treating cancer comprising administering any two different siRNAs from the group consisting of SEQ ID NOS: 14, 16, and 17. Included are the above methods of suppressing Ii wherein the cell is a cancer cell.

In a related embodiment, the invention includes a composition comprising a combination of siRNAs effective to inhibit Ii expression, wherein the combination is comprised of any two different siRNAs selected from SEQ ID NOS: 14, 16, and 17. The effective composition can be composed of any two different siRNAs from the group as all combinations were significantly more effective than single siRNAs. The composition of the invention includes the most effective combination demonstrated, that of SEQ ID NOS: 14 and 17.

A wide variety of delivery systems are available for use in delivering an siRNA of the present invention to a target cell in vitro and in vivo. An siRNA of the present invention may be introduced directly or indirectly into a cell in which Ii inhibition is desired. An siRNA may be directly introduced into a cell by, for example, injection. As such, it is an object of the invention to provide for a composition comprising an siRNA effective to inhibit Ii in injectable, dosage unit form. An siRNA of the present invention may be injected intravenously or subcutaneously as an example, for therapeutical use in conjunction with the methods and compositions of the present invention. Such treatment may include intermittent or continuous administration until therapeutically effective levels are achieved to inhibit Ii expression in the desired tissue.

Indirectly, an expressible DNA sequence or sequences encoding the siRNA may be introduced into a cell, and the siRNA thereafter transcribed from the DNA sequence or sequences. It is an object of the present invention, therefore, to provide for compositions comprising a DNA sequence or sequences which encode one or more siRNA effective to inhibit Ii expression. Expressible siRNA constructs, might be preferred to synthetic oligonucleotides for the following reasons. 1) Transfection of cells with RNA oligonucleotides can be more difficult than is transfection with DNA expression constructs. 2) Large scale synthesis of synthetic siRNA oligonucleotides is more expensive than is preparation of a DNA plasmid or other vector. 3) Expression of the construct (and hence the Ii suppressive activity) can be targeted to specific organs or tissues using tissue-specific promoters. 4) The activity of siRNA (whether synthetic or expressed from a genetic vector) is generally much higher than is the activity of reverse gene constructs. For these reasons, expressible siRNA constructs have greater potential benefit for in vivo use.

A DNA composition of the present invention comprises a first DNA sequence which encodes a first RNA sequence comprising a sense sequence of Ii and a second DNA sequence which encodes a second RNA sequence comprising the reverse complement of the sense sequence of Ii. The first and second RNA sequences, when hybridized, form an siRNA duplex capable of forming an RNA-induced silencing complex, the RNA-induced silencing complex being capable of inhibiting Ii expression. The first and second DNA sequences may be chemically synthesized or synthesized by PCR using appropriate primers to Ii. Alternatively, the DNA sequences may be obtained by recombinant manipulation using cloning technology, which is well known in the art. Once obtained, the DNA sequences may be purified, combined, and then introduced into a cell in which Ii inhibition is desired. Alternatively, the sequences may be contained in a single vector or separate vectors, and the vector or vectors introduced into the cell in which Ii inhibition is desired.

In another embodiment, the present invention includes a method of suppressing Ii in a cell comprising administering an expressible construct comprising a DNA sequence(s) which encode two different siRNAs selected from the group consisting of SEQ ID NOS: 14, 16, and 17. The construct can be made of one or more DNA sequences which together encode two different siRNAs from the group. As with the direct methods of suppressing Ii using siRNAs described above, the method of this embodiment includes the method where the cell in which Ii is suppressed is a cancer cell. Furthermore, the present invention includes methods of treating cancer comprising administering an expressible construct comprising a DNA sequence(s) which encodes two different siRNAs selected from the group consisting of SEQ ID NOS: 14, 16, and 17. Another embodiment included in the invention is a composition comprising a DNA sequence(s) which encodes two different siRNAs selected from the group consisting of SEQ ID NOS: 14, 16, and 17; wherein the siRNAs are effective to inhibit Ii expression.

Delivery systems available for use in delivering a DNA composition of the present invention to a target cell include, for example, viral and non-viral systems. Examples of suitable viral systems include, for example, adenoviral vectors, adeno-associated virus, lentivirus, poxvirus, retroviral vectors, vaccinia, herpes simplex virus, HIV, the minute virus of mice, hepatitis B virus and influenza virus. Non-viral delivery systems may also be used, for example using, uncomplexed DNA, DNA-liposome complexes, DNA-protein complexes and DNA-coated gold particles, bacterial vectors such as salmonella, and other technologies such as those involving VP22 transport protein, Co-X-gene, and replicon vectors.

The compositions of the present invention include a DNA sequence(s) which encodes any two different siRNAs selected from SEQ ID NOS: 14, 16, and 17 wherein the DNA sequence(s) is in a plasmid or a viral vector. The compositions administered in the Exemplification section were DNA sequences cloned into pSuppressorAdeno or pBudCE4.1 plasmids.

One option for expressing a nucleic acid sequence of interest in an animal cell is the adenovirus system. In the Exemplification section which follows, the use of an adenovirus system is specifically disclosed. Adenovirus possesses a double-stranded DNA genome, and replicates independently of host cell division. Adenoviral vectors offer a variety of advantages relative to alternative methods for introducing expressible constructs into cells. For example, adenoviral vectors are capable of transducing a broad spectrum of human tissues and high levels of gene expression can be obtained in dividing and nondividing cells. Adenoviral vectors are characterized by a relatively short duration of transgene expression due to immune system clearance and dilutional loss during target cell division. Several routes of administration can be used including intravenous, intrabiliary, intraperitoneal, intravesicular, intracranial and intrathecal injection, and direct injection of a target organ or tissue. Thus, it is recognized in the art that targeting based on anatomical boundaries is achievable.

The adenoviral genome encodes about 15 proteins and infection involves a fiber protein which binds to a cell surface receptor. This receptor interaction results in internalization of the virus. Viral DNA enters the nucleus of the infected cell and transcription is initiated in the absence of cell division. Expression and replication is under control of the E1A and E1B genes (see Horwitz, M. S., In Virology, 2.sup.nd ed., 1990, pp. 1723-1740). Removal of E1 genes renders the virus replication-incompetent. Adenoviral serotypes 2 and 5 have been extensively used for vector construction. Beft et al. (Proc. Nat. Acad. Sci. U.S.A. 91: 8802-8806 (1994)) have used an adenoviral type 5 vector system with deletions of the E1 and E3 adenoviral genes.

Adeno-associated virus (AAV) (Kotin, R. M., Hum. Gene Ther. 5: 793-801 (1994)) are single-stranded DNA, non-autonomous parvoviruses able to integrate into the genome of non-dividing cells of a very broad host range. AAV has not been shown to be associated with human disease and does not elicit an immune response. AAV has two distinct life cycle phases. Wild-type virus will infect a host cell, integrate and remain latent. In the presence of adenovirus, the lytic phase of the virus is induced, which depends on the expression of early adenoviral genes, and leads to active virus replication. The AAV genome is composed of two open reading frames (called rep and cap) flanked by inverted terminal repeat (ITR) sequences. The rep region encodes four proteins which mediate AAV replication, viral DNA transcription, and endonuclease functions used in host genome integration. The rep genes are the only AAV sequences required for viral replication. The cap sequence encodes structural proteins that form the viral capsid. The ITRs contain the viral origins of replication, provide encapsidation signals, and participate in viral DNA integration. Recombinant, replication-defective viruses that have been developed for gene therapy lack rep and cap sequences. Replication-defective AAV can be produced by co-transfecting the separated elements necessary for AAV replication into a permissive cell line. U.S. Pat. No. 4,797,368 contains relevant disclosure and such disclosure is incorporated herein by reference.

Retroviral vectors are useful for infecting dividing cells, and are composed of an RNA genome that is packaged in an envelope derived from host cell membrane and viral proteins. Retroviral gene expression involves a reverse transcription step in which its positive-strand RNA genome is employed as a template to direct the synthesis of double-stranded DNA, which is then integrated into the host cell DNA. The integrated provirus is able to use host cell machinery for gene expression.

Murine leukemia virus is a commonly employed retrovirus species (Miller et al., Methods Enzymol. 217: 581-599 (1993)). Retroviral vectors are typically constructed by deletion of the gag, pol, and env genes. The deletion of these sequences provides capacity for insertion of nucleic acid sequences of interest and eliminates the replicative functions of the virus. Genes encoding antibiotic resistance often are included as a means of selection. Promoter and enhancer functions also may be included, for example, to provide for tissue-specific expression following in vivo administration. Promoter and enhancer functions contained in long terminal repeats may also be used.

Such viruses, and modifications of such viruses which carry an exogenous nucleic acid sequence of interest, can only be produced in viral packaging cell lines. The packaging cell line may be constructed by stably inserting the deleted viral genes (gag, pol, and env) into the cell such that they reside on different chromosomes to prevent recombination. The packaging cell line is used to construct a producer cell line that will generate replication-defective retrovirus containing the nucleic acid sequence of interest by inserting the recombinant proviral DNA. Plasmid DNA containing the long terminal repeat sequences flanking a small portion of the gag gene that contains the encapsidation sequence and the genes of interest is transfected into the packaging cell line using standard techniques for DNA transfer and uptake (electroporation, calcium precipitation, etc.). Variants of this approach have been employed to decrease the likelihood of production of replication-competent virus (Jolly, D., Cancer Gene Therapy 1: 51-64 (1994)). The host cell range of the virus is determined by the envelope gene (env) and substitution of env genes with different cell specificities can be employed. Incorporation of appropriate ligands into the envelope protein may also be used for targeting.

Administration of recombinant retroviral vectors may be accomplished by any suitable technique. Such techniques include, for example, ex vivo transduction of patients' cells, direct injection of virus into tissue, and the administration of the retroviral producer cells. Ex vivo approaches require the isolation and maintenance in tissue culture of the patient's cells. In this context, a high ratio of viral particles to target cells can be achieved and thus improve the transduction efficiency (see, e.g., U.S. Pat. No. 5,399,346, the disclosure of which is incorporated herein by reference). U.S. Pat. No. 4,650,764 contains disclosure relevant to the use of retroviral expression systems and the disclosure of this referenced patent is also incorporated herein by reference.

In some cases direct introduction of virus in vivo is necessary or preferred. Retroviruses have been used to treat brain tumors wherein the ability of a retrovirus to infect only dividing cells (tumor cells) may be particularly advantageous.

The administration of a retrovirus producer cell line directly into a brain tumor in a patient has also been proposed (see e.g., Oldfield et al., Hum. Gene Ther. 4: 39-69 (1993)). Such a producer cell would survive within the brain tumor for a period of days, and would secrete retrovirus capable of transducing the surrounding brain tumor.

Pox virus-based systems for expression have been described (Moss and Flexner, Annu. Rev. Immunol 5: 305-324 (1987); Moss, B., In Virology, 1990, pp. 2079-2111). Vaccinia, for example, are large, enveloped DNA viruses that replicate in the cytoplasm of infected cells. Nondividing and dividing cells from many different tissues are infected, and gene expression from a nonintegrated genome is observed. Recombinant virus can be produced by inserting the transgene into a vaccinia-derived plasmid and transfecting this DNA into vaccinia-infected cells where homologous recombination leads to the virus production. A significant disadvantage is that it elicits a host immune response to the 150 to 200 virally encoded proteins making repeated administration problematic.

The herpes simplex virus is a large, double-stranded DNA virus that replicates in the nucleus of infected cells. This virus is adaptable for use in connection with exogenous nucleic acid sequences (see Kennedy and Steiner, Q. J. Med. 86: 697-702 (1993)). Advantages include a broad host cell range, infection of dividing and nondividing cells, and the fact that large sequences of foreign DNA can be inserted into the viral genome by homologous recombination. Disadvantages are the difficulty in rendering viral preparations free of replication-competent virus and a potent immune response. Deletion of the viral thymidine kinase gene renders the virus replication-defective in cells with low levels of thymidine kinase. Cells undergoing active cell division (e.g., tumor cells) possess sufficient thymidine kinase activity to allow replication.

A variety of other viruses, including HIV, the minute virus of mice, hepatitis B virus, and influenza virus, have been disclosed as vectors for gene transfer (see Jolly, D., Cancer Gene Therapy 1: 51-64 (1994)).

The compositions of the present invention include those effective to inhibit Ii expression which comprise DNA sequence(s) encoding two different siRNAs from SEQ ID NOS: 14, 16, and 17, wherein the DNA sequence(s) is in a viral vector. The viral vector may be selected from the group consisting of adenovirus, adeno-associated virus, lentivirus, poxvirus, influenza, and retrovirus.

Nonviral DNA delivery strategies are also applicable. These DNA delivery strategies relate to uncomplexed plasmid DNA, DNA-lipid complexes, DNA-liposome complexes, DNA-protein complexes, DNA-coated gold particles, and DNA-coated polylactide coglycolide particles. Purified nucleic acid can be injected directly into tissues and results in transient gene expression for example in muscle tissue, and is particularly effective in regenerating muscle (Wolff et al., Science 247:1465-1468 (1990)). Davis et al. (Hum. Gene Ther. 4: 733-740 (1993)) has published on direct injection of DNA into mature muscle (skeletal muscle is generally preferred).

Plasmid DNA on gold particles can be “fired” into cells (e.g. epidermis or melanoma) using a gene-gun. DNA is coprecipitated onto the gold particle and then fired using an electric spark or pressurized gas as propellant (Fynan et al., Proc. Natl. Acad. Sci. U.S.A. 90: 11478-11482 (1993)). Electroporation has also been used to enable transfer of DNA into solid tumors using electroporation probes employing multi-needle arrays and pulsed, rotating electric fields (Nishi et al., Cancer Res. 56: 1050-1055 (1996)). High efficiency gene transfer to subcutaneous tumors has been claimed with significant cell transfection enhancement and better distribution characteristics over intra-tumoral injection procedures.

Lipid-mediated transfections are preferred for both in vitro and in vivo transfections (Horton et al., J. Immunology 162: 6378 (1999)). Lipid-DNA complexes are formed by mixing DNA and lipid 1 to 5 minutes before injection, using commercially available lipids such as DMRIE-C reagent.

Liposomes work by surrounding hydrophilic molecules with hydrophobic molecules to facilitate cell entry. Liposomes are unilamellar or multilamellar spheres made from lipids. Lipid composition and manufacturing processes affect liposome structure. Other molecules can be incorporated into the lipid membranes. Liposomes can be anionic or cationic. Nicolau et al. (Proc. Natl. Acad. Sci. U.S.A. 80:1068-1072 (1983)) has published work relating to insulin expression from anionic liposomes injected into rats. Anionic liposomes mainly target the reticuloendothelial cells of the liver, unless otherwise targeted. Molecules can be incorporated into the surface of liposomes to alter their behavior, for example cell-selective delivery (Wu and Wu, J. Biol. Chem. 262: 4429-4432 (1987)).

Feigner et al. (Proc. Nat Acad. Sci. U.S.A. 84: 7413-7417 (1987)) has published work relating to cationic liposomes, demonstrated their binding of nucleic acids by electrostatic interactions, and shown cell entry. Intravenous injection of cationic liposomes leads to transgene expression in most organs on injection into the afferent blood supply to the organ. Cationic liposomes can be administered by aerosol to target lung epithelium (Brigham et al., Am. J. Med. Sci. 298: 278-281 (1989)). In vivo studies with cationic liposome transgene delivery have been published (see, e.g., Nabel et al., Rev. Hum. Gene Ther. 5: 79-92 (1994); Hyde et al., Nature 362: 250-255 (1993) and; Conary et al., J. Clin. Invest 93: 1834-1840 (1994)).

Microparticles are being studied as systems for delivery of DNA to phagocytic cells. Such approaches have been reported by Pangaea Pharmaceuticals. Such a DNA microencapsulation delivery system has been used to effect more efficient transduction of phagocytic cells, such as macrophages, which ingest the microspheres. The microspheres encapsulate plasmid DNA encoding potentially immunogenic peptides which, when expressed, lead to peptide display via MHC molecules on the cell surface which can stimulate immune response against such peptides and protein sequences which contain the same epitopes. This approach is presently aimed towards a potential role in anti-tumor and pathogen vaccine development but may have other possible gene therapy applications.

Natural viral coat proteins which are capable of homogeneous self-assembly into virus-like particles (VLPs) have also been used to package DNA for delivery. The major structural coat protein (VP1) of human polyoma virus can be expressed as a recombinant protein and is able to package plasmid DNA during self-assembly into a VLP. The resulting particles can be subsequently used to transduce various cell lines.

Improvements in DNA vectors have also been made and are likely applicable to many of the non-viral delivery systems. These include the use of supercoiled minicircles (which do not have bacterial origins of replication nor antibiotic resistance genes and thus are potentially safer as they exhibit a high level of biological containment), episomal expression vectors (replicating episomal expression systems where the plasmid amplifies within the nucleus but outside the chromosome and thus avoids genome integration events) and T7 systems (a strictly a cytoplasmic expression vector in which the vector itself expresses phage T7 RNA polymerase and the therapeutic gene is driven from a second T7 promoter, using the polymerase generated by the first promoter). Other, more general improvements to DNA vector technology include use of cis-acting elements to effect high levels of expression, sequences derived from alphoid repeat DNA to supply once-per-cell-cycle replication, and nuclear targeting sequences.

The methods of the present invention, of inhibiting Ii and/or treating cancer by administering an expressible construct comprising a DNA sequence(s) encoding two different siRNAs include an embodiment wherein the DNA sequence(s) is introduced into the cell by a method employing a mediator selected from the group consisting of cationic dendrimers, lipids, liposomes, gold particles, polylactide cogylcolide particles, and polyalkyloxide copolymers. Example 1 of the Exemplification section describes in detail how the DNA sequences were delivered to cells on gold particles.

In all embodiments of the invention, it is possible to provide one or more siRNAs as a single molecular construct (e.g., using a viral vector delivery system having a sufficient capacity to accept nucleic acid encoding both siRNAs). Additional sequences may be included in this single molecular construct. Alternatively, separate expression constructs may be used to carry each siRNA. In the case of separate constructs, delivered in an independent manner, the likelihood of a single antigen presenting cell taking up each of the two constructs is an issue of statistical probability. Furthermore, packaging more than one construct in a single viral particle has the utility of maximizing the therapeutically effective induction of Ii suppression relative to the synthesis of viral proteins to which is generated an immune response which is deleterious. Such an anti-viral immune response can for example limit the frequency with which such therapeutic interventions are possible.

Introduction by non-viral delivery systems requires specific consideration as well. By using non-viral delivery systems, uncomplexed DNA, DNA-liposome complexes, DNA-protein complexes, and DNA-coated gold particles can be delivered into cells. Each of these methods offers advantages and disadvantages which control selection for specific pathologies. The use of complexed DNA (e.g., DNA-liposome complexes, DNA-protein complexes, DNA-coated gold particles, and microencapsulation in polylactide cogylcolide particles) would tend to ensure delivery to a single cell of both nucleic acid sequences encoding inhibitors of Ii expression. Even if encoded by distinct molecular species, both species would tend to be delivered to a single cell because they are “packaged” (e.g., either encapsulated in a liposome, or coated onto a gold particle).

DNA-coated gold particles are commonly delivered by a ballistic method using the so-called “gene gun” technology. Using this technique, gold particles can be fired into the skin or muscle tissue and used to penetrate cells. Penetrated cells have been shown to express nucleic acid sequences introduced in this manner. Dendritic cells are naturally occurring antigen presenting cells which can be effectively transfected using this technique. Such expression constructs, when introduced into a single dendritic cell, for example, will result in the display of the antigenic epitope of interest on the surface of the antigen presenting cell in association with MHC Class II molecules. The display of the epitope/MHC Class II molecule complex on the surface of the antigen presenting cell will stimulate additional immune cells providing a heightened immune response.

Alternatively, when addressing a pathological condition having a defined anatomical location (e.g., primary tumors or some metastases of neoplastic diseases), direct injection into the defined anatomical site may be indicated. Such sites will tend to be enriched in antigen presenting cells such as dendritic cells. A tumor is an example of such a local site of introduction. If the constructs which inhibit Ii expression are taken up by a cell exhibiting a pathology (e.g., a tumor cell) the cell will display pathology-specific epitopes on its cell surface in association with MHC Class II molecules. These cells also will stimulate T helper cells and B lymphocytes.

As discussed above, the present invention relates to inhibition of Ii in a variety of animal cell types, either in vivo or ex vivo. Another embodiment of the present invention includes a mammalian cell containing any two different siRNAs selected from the group consisting of SEQ ID NOS: 14, 16, and 17. Another embodiment of the present invention includes a mammalian cell containing an expressible construct comprising a DNA sequence(s) which encode two different siRNAs selected from the group consisting of SEQ ID NOS: 14, 16, and 17. In either of the two preceding embodiments, the mammalian cell may be a cancer cell. Three types of cancer cells containing the combinations of siRNAs or containing expressible DNA constructs encoding said combinations were created in examples 1-4 of the Exemplification section. The mammalian cells of this invention include those wherein the DNA sequence(s) is in a plasmid or viral vector. The viral vector within the mammalian cell may be selected from the group consisting of adenovirus, adeno-associated virus, lentivirus, poxvirus, influenza, and retrovirus.

With respect to the naturally occurring antigen presenting cells, in vivo and ex vivo applications are included. In the present disclosure, the term “targeting” is sometimes used to describe the directing of an immune response toward an antigenic protein or a particular antigenic epitope within an antigenic protein. This immune response is characterized, in part, by the activation of T immunoregulatory cells, such as T helper cells or T suppressor cells, which may be variably Th1, or Th2, or Th3 cells, depending upon the context of the response. For example a Th1 response is a helper response with respect to development of a CTL response to a tumor antigen, which response leads to killing of tumor cells. A Th1 response to an allergen, however, may be functionally a suppressing response, with respect to immunodeviating the response to the allergen away from a Th2 response, which leads to production of pathogenic IgE antibodies. In addition, the concept of targeting includes, not only the initial portions of the immune response which are stimulated by the presentation of MHC Class II-presented epitopes which are novel or in increased amounts, but also those downstream effector responses which are induced or regulated by the initial actions on T immunoregulatory cells. Thus, for example, targeting includes the CTL-anticancer response or the immunoglobulin anti-viral response which may be initiated by the method of targeting taught herein.

Targeting includes the concept that the immune response is directed to an antigen, whether the antigen either is specified or is not known, nor even identifiable without undue experimentation. For example, targeting may be directed to a cell that may express a large number of antigens each of which may contribute to the generation of an immune response. What particular antigens within a cell participate in the immune response may vary from person to person depending upon the genetic makeup of the individuals. The susceptibility of the immune response to genetic factors has been well described. Consequently, in using the method of targeting for a useful therapeutic or diagnostic purpose, the specific antigenic components of the cell need not and often cannot be specified.

The process of targeting includes processes occurring either in vivo or in vitro. In vivo, for example, the activation of immunoregulatory T cells to antigen presented by MHC Class II-positive cells which are either tumor cells or dendritic cells may occur in either a non-tumor location or infiltrating a tumor. The expansion of the effector portion of the immune response likewise may occur either in vivo or in vitro. In the case of in vitro responses, products can be generated which may be reintroduced into the individual, or into another selected individual, to effect a therapeutic response. Examples, of such products include dendritic cell preparations, cytotoxic T cell preparations, and antibodies which might have been produced after cloning B cells from such an in vitro targeted culture, for example after the production of B cell hybridomas.

Toward this end, depending upon the therapeutic product desired for introduction into the individual from which peripheral blood mononuclear cells have been obtained, the original cultures might be fractionated to enrich for a desired cell population, for example, dendritic cells or T lymphocytes. In addition, the culture after the targeting process taught herein has been effected, may be fractionated for a desired cell population, for example, dendritic cells or T lymphocytes. Established methods are available for the fractionation of cells obtained from an individual either immediately after isolation and before the targeting process of this invention, or subsequently after that targeting process has been effected. Furthermore, established procedures are available for the introduction of such products into the individual from which peripheral blood mononuclear cells were originally obtained. To this end, the methods of this invention with respect to targeting are not limited to peripheral blood mononuclear cells, but include all cellular preparations which might be obtained from an individual including mucosal cells from the oropharynx or other regions, cells obtained after bronchial or gastric lavage, cells obtained by biopsy or excision from any organ, such as tumor tissues or normal tissues for example from liver, pancreas, prostate, skeletal muscle, fat, or skin.

In another embodiment, the present invention includes a method for targeting a type of cell of an individual for an immunological response wherein the type of cell is characterized by the expression of one or more identified or unknown antigen(s). The method comprises providing, in culture, peripheral blood mononuclear cells of the individual including antigen presenting cells, and introducing into the antigen presenting cells two different siRNAs selected from the group consisting of SEQ ID NOS: 14, 16, and 17. The siRNAs can be introduced either directly or indirectly into the cells, either will inhibit the expression of Ii. Another embodiment of the present invention includes the method for targeting a type of cell described above and further comprises reintroducing the antigen presenting cells containing the siRNAs into the individual for a therapeutic effect. The methods of the invention include embodiments wherein the targeted cell is a cancer cell.

With respect to ex vivo applications, tumor cells are isolated from the individual and an ex vivo culture is established. Such cultures can be established from an unselected population of malignant cells obtained from the individual, with or without separation from accompanying normal cells, or cells can be obtained as cell lines or clones from such cell lines. Alternatively, such cells are obtained from established malignant cell lines of unrelated patients or as explants of fresh malignant tissue (e.g., colon or ovarian carcinoma).

Ii suppressor siRNAs are introduced into the cultured cells, resulting in the desired MHC Class II molecules-associated presentation of tumor-specific or tumor-related antigenic epitopes. Cells treated in this manner are rendered replication incompetent (e.g., by irradiation or fixation), and used in a conventional immunization protocol (e.g., subcutaneous, intravenous, intraperitoneal or intramuscular immunization). In addition to whole cell formulations, other derivatives thereof may be used in the immunization formulation.

Although many tumor cells are MHC Class II molecule and Ii-negative, it is well-known that some tumors (for example, certain lymphomas, melanomas and adenocarcinomas, affecting, for example, breast, lung and colon) are MHC Class II molecule-positive and Ii-positive. In this subset which express MHC Class II molecules, the introduction of only an Ii suppressor should be adequate to achieve the desired immune stimulation. It will be recognized that the inclusion of an MHC Class II molecule inducer in such cells may serve to enhance the desired stimulation by increasing the likelihood of T helper cell interaction with MHC Class II molecules-associated antigen.

Both DNA and RNA vaccine viruses may contain a construct for expression of an siRNA leading to suppression of Ii protein expression in the infected cell. In the case of a DNA virus, such as vaccinia, the genes are under the control of classical mammalian promoters such as CMV, RSV, Ubc, EF-1α, and U6. In the case of RNA viruses, such as influenza, translations from the RNA of the inserted constructs are expressed by the influenza viral enzyme mediating RNA transcription and translation mechanisms. A vaccine virus, with the capacity to suppress expression of Ii protein in an infected cell, is targeted for cell types which already endogenously express Ii protein and MHC class II molecules. Such cell types include Langerhans cells of the skin, other dendritic cells in skin or in mucosal surfaces of the respiratory tract or gut, or which might have been mobilized from bone marrow, or obtained from bone marrow or spleens, macrophages of the peripheral blood or other bodily fluids such as exudative or transudative fluids arising or induced in abdominal, pleural, pericardial or other bodily cavities. Additional cell types include B cells, or B lineage leukemias and lymphomas, and cells which by activation have come to express MHC class II molecules and Ii protein, such as some subsets of T cells and transformed malignant or normal cells.

The construction of examples of these DNA or RNA viruses can be achieved with standard molecular biological techniques. The cDNA encoding Ii-specific siRNA can be introduced using standard molecular cloning methods into plasmids encoding vaccinia, canarypox, or other DNA viruses (Panicali D. Proc Natl Acad Sci USA. 1982; 16:4927-31). Intact vaccinia viral DNA as well as Ii-specific siRNA expression cassettes can be cloned into a vector flanked by viral sequences. Homologous recombination between the cloned Ii-specific siRNA expression cassettes can occur and novel viruses can be selected under the appropriate conditions (Panicali D. Proc Natl Acad Sci USA. 1982; 16:4927-31; Marti W R. Cell Immunol. 1997:179:146-52; Bertley F M N. J. Immunol. 2004; 172:3745-57). Recombinant RNA viruses can be similarly constructed using plasmids encoding viral cDNAs. A plasmid-based reverse genetics system for Influenza A virus has been developed (Pleschka S. J Virol 1996; 70:4188-92). This system uses plasmids containing a truncated human polymerase I promoter to express viral RNA. Ii-specific siRNA expression cassettes can be cloned into a plasmid encoding the influenza HA or NA gene. Plasmids encoding all 8 segments of the viral genome can be cotransfected into tissue cultured cells to recover infectious recombinant viruses that can be used for vaccination purposes. Alternatively, a recombinant plasmid encoding Ii-specific siRNAs can be transfected into a cell line infected with an influenza helper virus. Using a selection method, viruses containing the genetically engineered transfectant virus can be isolated (Palese P. J. Virol. 1996; 93:11354-8). Design and preparation of these various constructs, and their applications as vaccines, can be executed with the materials and methods of the following US patents. U.S. Pat. No. 5,976,552, U.S. Pat. No. 5,292,506, U.S. Pat. No. 4,826,687, U.S. Pat. No. 6,740,325, U.S. Pat. No. 6,651,655, U.S. Pat. No. 5,948,410, U.S. Pat. No. 5,824,536, U.S. Pat. No. 4,029,763, U.S. Pat. No. 4,009,258, U.S. Pat. No. 668,463, U.S. Pat. No. 667,611, U.S. Pat. No. 6,623,962, and U.S. Pat. No. 6,506,559.

Another embodiment of the present invention includes a method of suppressing Ii in a cell by administering an expressible construct comprising a DNA sequence(s) encoding two different siRNAs wherein the DNA sequence(s) which encode the siRNAs are operably linked to an RNA polymerase promoter. The method includes embodiments wherein the RNA polymerase promoter is CMV or EF-1α. The DNA sequences encoding siRNAs which suppressed Ii expression were linked to a CMV promoter in Examples 1-4 and to an EF-1α promoter in Examples 3-4, detailed in the Exemplification section which follows. The expressible constructs were successful at inhibiting Ii when driven by a CMV promoter in all cancer types tested. The methods of the present invention include those of suppressing Ii with an expressible DNA construct wherein the cell is a cancer cell, the promoter is CMV, and the cancer is AML, prostate cancer, or B cell lymphoma. When the promoter linked to the expressible construct was EF-1α, Ii was successfully suppressed in AML and prostate cancer. The methods of the present invention include those of suppressing Ii wherein the cell is a cancer cell, the promoter is EF-1α, and the cancer is AML or prostate cancer. The present invention also includes compositions comprising DNA sequence(s) which encode two different siRNAs effective to inhibit Ii expression wherein the DNA sequence(s) which are operably linked to an RNA polymerase III promoter, including a CMV or EF-1α promoter.

### EXEMPLIFICATION

### Inhibition of Ii in Human Cells with siRNA Plasmids

Ten Ii-siRNA constructs were generated and have been described in U.S. patent application Ser. No. 10/999,208. These single siRNA constructs were tested for inhibition of Ii expression in the non-cancerous Human Embryonic Kidney (HEK) 293 cell line.

Design of siRNA(Ii) Constructs

Ten siRNA(Ii) constructs were designed, with the oligonucleotides used in their construction presented in Table 1. The constructs were made with the pSuppressorAdeno plasmid (Imgenex. San Diego, Calif.), which was designed specifically for cloning of siRNAs. The plasmid contains a CMV promoter optimized for siRNA expression, provides a convenient cloning site for inserting siRNA sequences, and permits delivery to a wide variety of cells. Further, this plasmid can also be used toward construction of a recombinant adenovirus containing the siRNA-expressing construct. Two approaches were followed in the design of these siRNA(Ii) constructs. First, the Imgenex computer program was used to predict 5 constructs (constructs 11-15 in Table 1). This program identifies RNA sequences that have a base composition likely to hybridize to the Ii RNA (i.e., appropriate G-C content, etc.). The resulting 5 siRNA(Ii) constructs are expected to be potent inhibitors if they actually hybridize with Ii mRNA. However, since the tertiary structure of any given mRNA is difficult to predict, such computer-designed siRNA(Ii) constructs might not be found experimentally to be accessible to Ii mRNA. Therefore, a second approach was also used in the design of 5 additional constructs (constructs 16-20 in Table 1). Previous data on the use of Ii-RGC to inhibit expression of Ii protein revealed that some Ii antisense oligonucleotides (Qiu Cancer 1 mm Immunother. 48:499-506 (1999) (Xu U.S. Pat. No. 6,368,855) and Ii-reverse gene constructs (RGC; Lu Cancer Immunol Immunother. 52: 592-598 (2003)) (U.S. application Ser. No. 10/127,347) hybridize to the first 400 bp of human Ii mRNA, with potent consequent inhibition of Ii protein expression. One can deduce from these data, that this region of human Ii mRNA should be largely accessible to siRNA constructs. This proposal, furthermore, is consistent with the data in the literature that the mRNA region containing the AUG site starting translation is generally a sensitive region for antisense constructs to bind to mRNA. Therefore, by inspection, another 5 siRNA constructs were designed to hybridize to sections of Ii mRNA within the first 400 bp of human Ii mRNA around the AUG start site. Because there are two AUGs at the beginning of the human Ii mRNA, both of which appear to be functional translation start sites, siRNA sequences were designed to target both of these sites. Specifically, two overlapping sequences were designed around the first AUG and three overlapping siRNA sequences were designed around the second AUG. While these 5 siRNA (Ii) sequences might not have optimal annealing parameters, they can be expected to hybridize with Ii RNA. All sequences were designed with a short loop sequence to allow for hairpin formation of the expressed siRNA sequences. The formation of a hairpin results in a functional double-stranded siRNA. The requirement for double-stranded RNA in forming the RNA-induced silencing complex (RISC) that interacts with and cleaves target mRNA has been clearly demonstrated (Nature Reviews Genetics 2:110-119, 2001).

A double-stranded oligonucleotide is created by annealing two oligonucleotides coding for shRNA (short hairpin RNA) respectively for sense and complementary strands as indicated above. The annealed oligonucleotides will have “tcga” (shown above) and “gatc” overhangs to assist cloning into the linearized pSuppressorAdeno vectors. In Table 1 below, the sense sequence is single-underlined. The loop sequence is bold. The inverted sequence is double-underlined.

TABLE 1
Structure of 10 SiRNA constructs

| SEQ | | |
|---|---|---|
| ID NO. | Position | sequences |
| 11 | 1-21 | 5′tcgattcccagatgcacaggaggagatcgatctcctcctgtgcatctgggaattttt |
| 12 | 8-28 | 5′tcgaatgcacaggaggagaagcaggatcgatcctgcttctcctcctgtgcatttttt |
| 13 | 47-67 | 5′tcgaaagccagtcatggatgaccagatcgatctggtcatccatgactggcttttttt |
| 14 | 56-76 | 5′tcgaatggatgaccagcgcgaccttatcgataaggtcgcgctggtcatccatttttt |
| 15 | 84-104 | 5′tcgacaatgagcaactgcccatgctatcgatagcatgggcagttgctcattgttttt |
| 16 | 267-287 | 5′tcgacctgcagctggagaacctgcgatcgatcgcaggttctccagctgcaggttttt |
| 17 | 312-332 | 5′tcgagcctgtgagcaagatgcgcatatcgatatgcgcatcttgctcacaggcttttt |
| 18 | 396-416 | 5′tcgatgccaccaagtatggcaacatatcgatatgttgccatacttggtggcattttt |
| 19 | 414-434 | 5′tcgacatgacagaggaccatgtgatatcgatatcacatggtcctctgtcatgttttt |
| 20 | 501-521 | 5′tcgacctgagacaccttaagaacacatcgatgtgttcttaaggtgtctcaggttttt |

Creation of siRNA(Ii) Constructs

Ten siRNA(Ii) constructs were created by cloning the above sequences into pSuppressorAdeno plasmid (Imgenex, San Diego, Calif.) using Sal1 and Xba1 enzyme sites, according to standard molecular biological techniques. Cells of the 293 human kidney line (ATCC Number CRL-1573) were co-transfected with human Ii cDNA gene plasmid (0.18 μg) with each of these Ii siRNA constructs (0.82 μg). Several active Ii siRNA construct(s) were defined.

### Ii-RNAi Involved Ii Suppression in Tumor Cells

### Example 1

### Ii Inhibition in Raji Cells by Ii-siRNA Constructs

For the instant invention, the ten Ii-siRNA constructs, listed in Table 1 above, were used to transfect a human lymphoma cell line, Raji cells. The gene gun delivery method was used to transfect the Ii-siRNA constructs into Raji cells. Ii-siRNA sequences were cloned into a pSuppressorAdeno vector (Imgenex, San Diego) driven by a CMV promoter (refer as CMV/Ii-siRNA). The plasmid DNA was precipitated onto gold microparticles. Gold microcarriers (0.5 mg of 1 μm gold microparticles for one cartridge) were suspended by sonication in 100 μl of 0.1 M spermidine. The indicated amount of plasmid DNA (depending on the desired number of cartridges) at a concentration of 1 mg/ml in endotoxin-free water was added and sonicated and 200 μl of 1 M CaCl₂ was added drop wise. This gold-DNA mixture was allowed to stand for 10 min before being washed 3 times with 1 ml of 100% ethanol. After the final wash, the pellet was re-suspended in appropriate volume (depending on the desired quantity of gold microparticles) of 0.02 mg/ml polyvinylpyrrolidone (PVP) in 100% ethanol, transferred to a 15 ml tube. The result was a microcarrier loading quantity (MLQ) of 0.5 mg of gold per cartridge and a variable DNA loading ratio (DLR). One ml of DNA/microcarrier suspension produced 17 coated 0.5-inch cartridges, which were stored overnight at 4° C. with desiccant prior to use. For transfecting Raji cells, 106 Raji cells in 20 μl medium were smeared onto a tissue culture dish in an area about 0.8 cm in diameter and then subjected to a gene gun shooting with one 0.5-inch cartridge using a helium pressure of 350-400 psi. After transfection, the cells were further cultured for another 36-48 hours. The cells were then harvested, washed, and stained with anti-HLA-DR and anti-human Ii monoclonal antibodies and analyzed by flow cytometry to determine the percentage of HLA-DR+/Ii− cells. The Ii inhibition in Raji cells is shown in Table 2. From Table 2, one can see that Ii-siRNA sequences #14, #16, and #17 are active in inhibiting Ii expression in Raji cells. The inhibition is Ii-specific, since MHC class II molecule presentation is not apparently influenced by these Ii-siRNA constructs.

TABLE 2
Ii inhibition by CMV/Ii-siRNA plasmids in Raji cells. The
experimental procedures are described in the text and the
table shows the Fluorescence-Activated Cell-Sorting (FACS)
results of the experiment. Numbers are percents.

| Ii-siRNA constructs | MHC class II+ | Ii+ | Ii inhibition |
|---|---|---|---|
| Negative control | 0 | 0.5 | 0 |
| Empty plasmid | 99.5 | 97.5 | 0 |
| Sequence #11 | 99 | 97.3 | 0 |
| Sequence #12 | 98.3 | 96.1 | 0 |
| Sequence #13 | 99 | 97 | 0 |
| Sequence #14 | 98.5 | 70.5 | 27 |
| Sequence #15 | 99 | 97.8 | 0 |
| Sequence #16 | 98.6 | 70.1 | 27.4 |
| Sequence #17 | 99.1 | 65.2 | 32.5 |
| Sequence #18 | 99 | 98.1 | 0 |
| Sequence #19 | 98.2 | 98.2 | 0 |
| Sequence #20 | 99 | 97.5 | 0 |

### Example 2

### Ii Inhibition in Raji Cells by Single or Combination Use of Ii-siRNA Constructs

In another experiment, the active CMV/Ii-siRNA constructs (containing sequence #14, #16, and #17 in Table 2) were further used separately (1 μg/transfection) or in combination (0.5 μg+0.5 μg/transfection) to inhibit Ii expression in Raji cells. The plasmid DNA coating onto gold microparticles and transfection procedures were the same as those for the experiment of Table 2. Raji cells were harvested 36 to 48 hours after transfection, washed, and stained with anti-HLA-DR and anti-human Ii antibodies. The results are shown in Table 3. From Table 3, one can see that the use of a combination of plasmids that contain sequences #14 and #17 gave the most profound Ii inhibition in Raji cells, indicating that the use of a combination of Ii-siRNA sequences targeted at different sites of the Ii gene produces more effective Ii inhibition than the use of a single Ii-siRNA construct. The use of a combination of plasmids that contain sequences #14 and #16 or that contain sequences #16 and #17 gave less Ii inhibition compared to the use of the combination of plasmids that contain sequences #14 and #17, indicating that the latter combination is the best combination.

TABLE 3
Ii inhibition in Raji cells (B-cell lymphoma) by CMV/Ii-siRNA constructs
(14, 16, and 17 = plasmids containing those sequences, used
separately or in combination as indicated). The numbers are percentages
calculated from FACS figures, cut and weighted.

| siRNA constructs | Ii negative cells | MHC class II negative cells |
|---|---|---|
| Negative control | 98 | 100 |
| Positive control | 1 | 1 |
| Empty plasmid (1 μg) | 1 | 1 |
| 14 (1 μg) | 35 | 5 |
| 16 (1 μg) | 20 | 2 |
| 17 (1 μg) | 35 | 2 |
| 14 + 16 (0.5 + 0.5 μg) | 47 | 2 |
| 14 + 17 (0.5 + 0.5 μg) | 65 | 2 |
| 16 + 17 (0.5 + 0.5 μg) | 44 | 3 |

### Example 3

### Ii Inhibition in KG-1 (AML) Cells by CMV/Ii-siRNA and EF-1α/siRNA Constructs

The two most active (in Raji cells) Ii-siRNA sequences, #14 and #17 (see Table 2), were further cloned into a pBudCE4.1 plasmid (Invitrogen, CA) to be driven by an EF-1α promoter (refer as EF-1α/Ii-siRNA). To do this, two pairs of oligonucleotides were first annealed into 2 double-strand DNA sequences that are equivalent to DNA sequences of #14 and #17 (Table 2) with overhead Bgl II and Not I cloning sites. The Ii-siRNA sequences were driven by an EF-1α promoter. The resulting plasmids were confirmed by sequencing. KG-1 cells (human acute myelogenous leukemia (AML) cell line) were used to examine the activity of two active Ii-siRNA plasmids driven by either a CMV or EF-1α promoter. The procedures for plasmid coating onto gold microparticles and the procedures for gene gun involved transfection were the same as for the transfection of Raji cells. After transfection, the KG-1 cells were further cultured for 36 to 48 hours and cells were harvested, washed and stained with an anti-human Ii monoclonal antibodies and analyzed by flow cytometry to determine the percentage of Ii-negative cells. As indicated in Table 4, CMV/Ii-siRNA and EF-1α/Ii-siRNA constructs were almost equally active in KG-1 cells. The difference may be a reflection of variation in coating and transfection. The CMV driven plasmids were pSuppressorAdeno vectors, while the EF-1α driven plasmids were PBudCE4.1 vectors.

TABLE 4
Ii inhibition, relative to the positive control, by siRNA plasmids
in KG-1 cells. KG-1 cells were transfected with CMV/p4 (p4 = plasmid
containing sequence #14) plus CMV/p7 (p7 = plasmid containing
sequence #17) or EF-1α/p4 plus EF-1α/p7 (total 1 microgram).
The CMV/Ii-siRNA and EF-1α/siRNA gold microparticles used
in this experiment were prepared on different days to test the
repeatability.

| condition | % of Ii-positive cells | % of Ii inhibition |
|---|---|---|
| (−) | 1.59 | — |
| (+) | 85.28 | — |
| CMV/p4 + CMV/p7 | 75.83 | 11.1 |
| CMV/p4 + CMV/p7 | 74.83 | 12.3 |
| EF-1α/p4 + EF-1α/p7 | 72.31 | 14.3 |
| EF-1α/p4 + EF-1α/p7 | 73.62 | 13.7 |

### Example 4

### Ii Inhibition in PC-3 Cells by CMV/Ii-siRNA and EF-1α/siRNA Constructs

The activity of CMV/Ii-siRNA and EF-1α/Ii-siRNA constructs was further tested in prostate cancer cells. PC-3 prostate cancer cells were transfected with CMV/p7 (p7=sequence #17) and EF-1α/P7 plasmids by the Lipofectamine® 2000 (Invitrogen, CA) method according to the manufacturer's instruction. The result (Table 5) demonstrates that in PC-3 cells, the CMV promoter is much more active than the EF-1α promoter (compare CMV/p7 with EF-1α/p7). This result indicates that the activity of the promoter is cell type-specific. The overall data of this study indicate that the CMV promoter is an active promoter in all type of cells that have been tested. The EF-1α promoter is also active in all type of cells tested but the activity of EF-1α promoter in prostate cancer cells is much lower than the CMV promoter.

TABLE 5
Ii inhibition in PC-3 prostate cancer cells by CMV/p7 and EF-1α/p7
(p7 = plasmid containing sequence #17). PC-3 cells
were transfected by Ii-siRNA constructs with the Lipofectamine ® 2000
method and harvested and stained with anti-human Ii antibody
and analyzed by FACS.

| Condition | % of Ii-positive cells | % of Ii inhibition |
|---|---|---|
| (−) | 3.13 | — |
| (+) | 51.5 | — |
| Lipid only | 61.0 | — |
| Empty plasmid | 54.5 | — |
| CMV/p7 | 15.9 | 68.2 |
| EF-1α/p7 | 24.7 | 52.1 |

1. A method of suppressing Ii in a cell comprising administering any two different siRNAs selected from the group consisting of SEQ ID NOS: 4, 6, and 7.

2. The method of claim 1 wherein the two different siRNAs selected are SEQ ID NOS: 4 and 7.

3. A method of suppressing Ii in a cell comprising administering an expressible construct comprising a DNA sequence or sequences which encode two different siRNAs selected from the group consisting of SEQ ID NOS: 4, 6, and 7.

4. The method of claim 3 wherein the DNA sequence(s) which encode the siRNAs are operably linked to an RNA polymerase promoter.

5. The method of claim 4 wherein in the RNA polymerase promoter is CMV or EF-1α.

6. The method of claim 1 or 3 wherein the cell is a cancer cell.

7. The method of claim 5 wherein the cell is a cancer cell, the promoter is CMV, and the cancer is AML, prostate cancer, or B cell lymphoma.

8. The method of claim 5 wherein the cell is a cancer cell, the promoter is EF-1α, and the cancer is AML or prostate cancer.

9. A method of treating cancer comprising administering any two different siRNAs from the group consisting of SEQ ID NOS: 4, 6, and 7.

10. A method of treating cancer comprising administering an expressible construct comprising a DNA sequence or sequences which encode two different siRNAs selected from the group consisting of SEQ ID NOS: 4, 6, and 7.

11. The method of claim 3 or 10 wherein the DNA sequence(s) encoding the siRNAs is introduced into the cell by a method employing a mediator selected from the group consisting of cationic dendrimers, lipids, liposomes, gold particles, polylactide cogylcolide particles, and polyalkyloxide copolymers.

12. A composition comprising a combination of siRNAs effective to inhibit Ii expression, wherein said combination is comprised of any two different siRNAs selected from the group consisting of SEQ ID NOS: 4, 6, and 7.

13. The composition of claim 12, wherein the two different siRNAs are SEQ ID NOS: 4 and 7.

14. A composition comprising a DNA sequence or sequences which encode two different siRNAs selected from the group consisting of SEQ ID NOS: 4, 6, and 7; wherein the siRNAs are effective to inhibit Ii expression.

15. The composition of claim 14 wherein the DNA sequence or sequences which encode the siRNAs are operably linked to an RNA polymerase III promoter.

16. The composition of claim 15 wherein the promoter is a CMV or EF-1α promoter.

17. A mammalian cell containing any two different siRNAs selected from the group consisting of SEQ ID NOS: 4, 6, and 7.

18. A mammalian cell containing an expressible construct comprising a DNA sequence or sequences which encode two different siRNAs selected from the group consisting of SEQ ID NOS: 4, 6, and 7.

19. The mammalian cell of claim 17 or 18 wherein the cell is a cancer cell.

20. A method for targeting a type of cell of an individual for an immunological response, the type of cell being characterized by the expression of one or more identified or unknown antigen(s), the method comprising:
a) providing, in culture, peripheral blood mononuclear cells of the individual including antigen presenting cells; and
b) introducing into the antigen presenting cells of the culture of step a), two different siRNAs selected from the group consisting of SEQ ID NOS: 4, 6, and 7; wherein the siRNAs are introduced either directly or indirectly into the cells, thereby inhibiting expression of Ii.

21. The method of claim 20 further comprising reintroducing the cells of step b) into the individual for a therapeutic effect.

22. The method of claim 20 or 21 wherein the type of cell being targeted is a cancer cell.

23. The composition of claim 14 wherein the DNA sequence(s) is in a plasmid vector.

24. The composition of claim 14 wherein the DNA sequence(s) is in a viral vector.

25. The mammalian cell of claim 18 wherein the DNA sequence(s) is in a plasmid vector.

26. The mammalian cell of claim 18 wherein the DNA sequence(s) is in a viral vector.

27. The composition of claim 24 wherein the viral vector is selected from the group consisting of adenovirus, adeno-associated virus, lentivirus, poxvirus, influenza, and retrovirus.

28. The mammalian cell of claim 26 wherein the viral vector is selected from the group consisting of adenovirus, adeno-associated virus, lentivirus, poxvirus, influenza, and retrovirus.
